(19) United States
(12) Patent Application Publication
(10) Pub. No.: US 2013/0178740 A1
(43) Pub. Date: Jul. 11, 2013

(54) METHOD AND APPARATUS FOR PROVIDING ULTRASOUND IMAGE

(71) Applicant: Song-yi HAN, Gangwon-do (KR)

(72) Inventor: Song-yi HAN, Gangwon-do (KR)

(73) Assignee: SAMSUNG MEDISON CO., LTD.

(21) Appl. No.: 13/547,526

(22) Filed: Jul. 12, 2012

(30) Foreign Application Priority Data
Jan. 5, 2012 (KR) 10-2012-0001549

(51) Int. Cl. A61B 8/14

(52) U.S. Cl. 600/443

(57) ABSTRACT

A method of providing an ultrasound image includes: obtaining an ultrasound image of an object; detecting a plurality of layers having different sound speeds based on the ultrasound image; and displaying information about boundaries of the plurality of layers.

### CROSS-REFERENCE TO RELATED PATENT APPLICATION

This application claims the benefit of Korean Patent Application No. 10-2012-0001549, filed on Jan. 5, 2012, in the Korean Intellectual Property Office, the disclosure of which is incorporated herein in its entirety by reference.

### BACKGROUND OF THE INVENTION

1. Field of the Invention

The present invention relates to a method and apparatus for providing an ultrasound image which may display information about boundaries of a plurality of layers having different sound speeds.

2. Description of the Related Art

Ultrasound diagnostic devices transmit an ultrasound signal to a predetermined portion of an interior of a human body to be diagnosed from a surface of the human body and obtain an image of blood flow or a tomogram of a soft tissue by using information of the ultrasound signal reflected from the predetermined portion.

Such ultrasonic diagnostic devices have advantages in that they are designed to be small and have low cost and may display images in real time. Also, since ultrasound diagnostic devices are very safe because there is no risk of X-ray radiation, ultrasound diagnostic devices are widely used along with other image diagnostic devices such as X-ray diagnostic devices, computerized tomography (CT) scanners, magnetic resonance imaging (MRI) devices, and nuclear medicine diagnostic devices.

In general, ultrasonic diagnostic devices use an average sound speed (i.e., 1540 m/s) of a soft tissue in order to obtain an ultrasound image. However, for an obese or well-muscled patient whose fat layer has a sound speed of 1460 m/s or muscle layer has a sound speed of 1600 m/s, his/her sound speed is different from the average sound speed, thereby degrading the quality of an ultrasound image.

In order to solve this problem, phase-aberration correction that is performed on channel radio frequency (RF) data and sound speed correction that is performed on compressed data and measures a quality factor have been studied. However, such technologies are complex and require a long processing time because images are repeatedly obtained.

### SUMMARY OF THE INVENTION

According to an aspect of the present invention, there is provided a method of providing an ultrasound image, the method including obtaining an ultrasound image of an object; detecting a plurality of layers having different sound speeds based on the ultrasound image; and displaying information about boundaries of the plurality of layers.

The obtaining of the ultrasound image may include: receiving ultrasound echo signals from the object; and generating the ultrasound image by applying a delay curve to the ultrasound echo signals.

The detecting of the plurality of layers having the different sound speeds may include detecting the plurality of layers having the different sound speeds based on the ultrasound echo signals received from the object.

The detecting of the plurality of layers having the different sound speeds may include detecting the plurality of layers having the different sound speeds based on a brightness difference of the ultrasound image.

The detecting of the plurality of layers having the different sound speeds may include: receiving location information of the plurality of layers having the different sound speeds from the outside; and detecting the plurality of layers having the different sound speeds by further considering the location information.

The detecting of the plurality of layers having the different sound speeds may include: receiving information about a diagnosed portion from the outside; and detecting the plurality of layers having the different sound speeds by further considering the information about the diagnosed portion.

The method may further include: correcting the ultrasound image based on information about the different sound speeds of the plurality of layers to obtain a corrected ultrasound image; and displaying the corrected ultrasound image.

The correcting of the ultrasound image may include adjusting a delay curve based on the information about the different sound speeds of the plurality of layers.

According to another aspect of the present invention, there is provided an apparatus for providing an ultrasound image, the apparatus including: an image obtaining unit that obtains an ultrasound image of an object; a detecting unit that detects a plurality of layers having different sound speeds based on the ultrasound image; a display unit that displays information about boundaries of the plurality of layers; and a control unit that controls the image obtaining unit, the detecting unit, and the display unit.

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram illustrating an apparatus for providing an ultrasound image, according to an embodiment of the present invention;

FIG. 2 is a detailed block diagram illustrating the apparatus of FIG. 1;

FIG. 3 is a flowchart illustrating a method of providing an ultrasound image, according to an embodiment of the present invention;

FIG. 4 is a table showing sound speeds of human tissues, according to an embodiment of the present invention;

FIG. 5 is a flowchart illustrating a method of correcting an ultrasound image, according to an embodiment of the present invention;

FIG. 6 is a graph illustrating a delay curve according to a sound speed, according to an embodiment of the present invention;

FIG. 7 is an image illustrating a boundary between a plurality of layers having different sound speeds, according to an embodiment of the present invention;

FIG. 8 is an image manually illustrating a boundary between a plurality of layers having different sound speeds, according to an embodiment of the present invention;

FIG. 9 is an image semi-automatically illustrating a boundary between a plurality of layers having different sound speeds, according to an embodiment of the present invention; and

FIG. 10 is an image automatically illustrating a boundary between a plurality of layers having different sound speeds, according to an embodiment of the present invention.

### DETAILED DESCRIPTION OF THE INVENTION

The present invention will now be described more fully with reference to the accompanying drawings, in which exemplary embodiments of the invention are shown.

Terms used hereinafter are used considering the functions in the present invention and may be changed according to a user's or operator's intention, usual practice, or development of new technology. Also, some terms may be arbitrarily selected by the applicant, and in this case, the meaning of the terms will be explained in detail. Accordingly, the terms will be defined based on the entire content of the description of the present invention.

Unless otherwise noted, the term “comprise” or variations such as “comprises” or “comprising” is understood to mean “includes, but is not limited to” so that other elements that are not explicitly mentioned may also be included. Also, the term “unit” or “module” means a unit of performing at least one function and may operate by using hardware or software or a combination of hardware and software.

The term “ultrasound image” herein means an image of an object obtained by using ultrasound. Here, the ‘object’ may be understood to be part of a human body. For example, the object may be an organ of a human body, such as a liver, a heart, a womb, a brain, a breast, or an abdomen, or a fetus.

Throughout the specification, the term ‘user’ may be a medical professional, e.g., a doctor, a nurse, a clinical pathologist, or a medical imaging professional, but is not limited thereto.

Hereinafter, the present invention will be described more fully with reference to the accompanying drawings, in which exemplary embodiments of the invention are shown. The invention may, however, be embodied in many different forms and should not be construed as being limited to the embodiments set forth herein. When publicly known techniques or structures related to the present invention may unnecessarily make the present invention unclear, the detailed description will be omitted. Similar elements are denoted by similar reference numerals throughout.

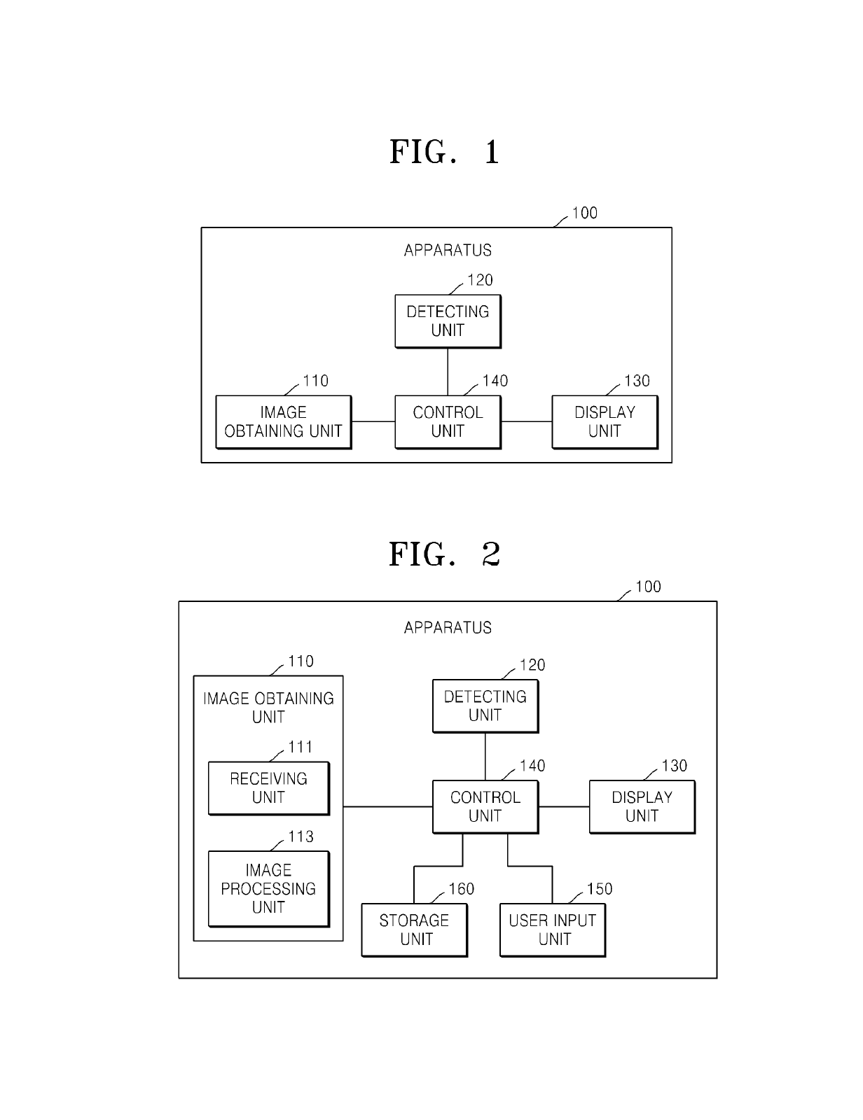

FIG. 1 is a block diagram illustrating an apparatus 100 for providing an ultrasound image, according to an embodiment of the present invention.

FIG. 2 is a detailed block diagram of the apparatus 100 of FIG. 1.

The apparatus 100 is a device for obtaining an ultrasound image from an object by using ultrasound and displaying the ultrasound image to a user.

The apparatus 100 may be realized in various ways. For example, the apparatus 100 may be realized as a fixed terminal or a mobile terminal. Examples of the mobile terminal may include a laptop computer, a personal digital assistant (PDA), and a tablet personal computer (PC).

Referring to FIGS. 1 and 2, the apparatus 100 may include an image obtaining unit 110, a detecting unit 120, a display unit 130, a control unit 140, a user input unit 150, and a storage unit 160. However, the elements of the apparatus 100 illustrated in FIGS. 1 and 2 are not limited thereto. The apparatus 100 may include more or less elements than those illustrated in FIG. 1.

The image obtaining unit 110 may obtain an ultrasound image of an object. The image obtaining unit 110 may receive an ultrasound image from the outside or may directly create an ultrasound image based on an ultrasound echo signal received from the object.

The image obtaining unit 110 may obtain a two-dimensional (2D) or three-dimensional (3D) ultrasound image.

The image obtaining unit 110 may include an ultrasound transmitting unit (not shown), a receiving unit 111, and an image processing unit 113. The ultrasound transmitting unit may include a transducer array. Also, the transducer array may include piezoelectric elements arranged in a linear array or a convex array.

The ultrasound transmitting unit may output an ultrasound signal by activating the piezoelectric elements in units of groups. That is, the ultrasound transmitting unit may transmit an ultrasound beam, which is generated when appropriately delayed pulse signals are transmitted, to the object along a scan line.

The receiving unit 111 may receive an ultrasound echo signal. When an ultrasound signal is output from the transducer array to the object, the receiving unit 111 may receive a plurality of ultrasound echo signals corresponding to a plurality of elements.

The image processing unit 113 may generate an ultrasound image by applying a delay curve to the ultrasound echo signals. That is, the image processing unit 113 focuses the plurality of ultrasound echo signals by using the delay curve.

When the apparatus 100 receives the plurality of ultrasound echo signals corresponding to the plurality of elements, since times when the ultrasound echo signals are received vary according to coordinates of the elements, a time delay occurs. In this case, the term “delay curve” refers to a travel-time curve of an ultrasound echo signal according to coordinates of each element. The delay curve varies according to a sound speed.

The detecting unit 120 may detect a plurality of layers having different sound speeds according to the ultrasound image obtained by the image obtaining unit 110. The detecting unit 120 may detect the plurality of layers having the different sound speeds based on the ultrasound echo signals received from the object.

The term “layer” herein is a layer of a human tissue which is included in the object and has a predetermined sound speed. For example, examples of the layer may include a fat layer, a muscle layer, a blood layer, a lung layer, and a liver layer.

Whenever an ultrasound beam is incident from a boundary surface between two materials having different acoustic impedances, part of the ultrasound beam is reflected and part of the ultrasound beam is transmitted. An amplitude of the reflected ultrasound beam varies according to an impedance difference at the boundary surface between the two materials. An acoustic impedance is obtained by multiplying a density by a sound speed.

Table 1 shows acoustic impedances in some tissues, air and water.

| Tissue | Impedance (Rayls) |
|---|---|
| Air | 0.0004 × 106 |
| Lung | 0.18 × 106 |
| Fat | 1.34 × 106 |
| Water | 1.48 × 106 |
| Liver | 1.65 × 106 |
| Blood | 1.65 × 106 |
| Kidney | 1.63 × 106 |
| Muscle | 1.71 × 106 |
| Skull bone | 7.8 × 106 |

Accordingly, the detecting unit 120 may detect the plurality of layers having the different sound speeds by analyzing the ultrasound echo signals, particularly, amplitudes of the ultrasound echo signals, received from the object.

Alternatively, the detecting unit 120 may detect the plurality of layers having the different sound speeds based on a brightness difference of the ultrasound image obtained by the image obtaining unit 110.

As an acoustic impedance difference increases, strengths of ultrasound echo signals increase. As the strengths of the ultrasound echo signals increase, a brightness of an ultrasound image increases.

Accordingly, the detecting unit 120 may detect the plurality of layers having the different sound speeds based on a brightness difference of the ultrasound image.

Alternatively, the detecting unit 120 may detect the plurality of layers having the different sound speeds by considering location information of the plurality of layers input from the outside. The location information may be information about boundary regions of the plurality of layers having the different sound speeds input from the outside.

For example, a user may set the boundary regions of the plurality of layers having the different sound speeds based on a displayed ultrasound image. In this case, the detecting unit 120 detects the plurality of layers having the different sound speeds by considering the boundary regions set by the user.

The detecting unit 120 may detect the plurality of layers having the different sound speeds by further considering information about a diagnosed portion (e.g., an applied portion) input from the outside.

For example, if the diagnosed portion consists of two layers, the detecting unit 120 may detect the two layers having different sound speeds, and if the diagnosed portion consists of three layers, the detecting unit 120 may detect the three layers having different sound speeds.

The display unit 130 may display and output information processed by the apparatus 100. For example, the display unit 130 may display the ultrasound image. In this case, the display unit 130 may display the ultrasound image in at least one mode selected from the group consisting of a brightness (B) mode, a color (C) mode, a motion (M) mode, a pulsed-wave (PM) mode, a continuous wave(CW) mode, a two-dimensional (2D) mode, and a three-dimensional(3D) mode.

The display unit 130 may display information about boundaries of the plurality of layers. Examples of the information about the boundaries of the plurality of layers may include information about boundary lines, boundary colors, and boundary coordinates.

The display unit 130 may display the ultrasound image that is corrected based on information about the different sound speeds of the plurality of layers.

If the display unit 130 is combined with a touchpad to act as a touch screen, the display unit 130 may be used as an input device as well as an output device. The display unit 130 may include at least one selected from the group consisting of a thin film transistor-liquid crystal display (TFT-LCD), an organic light-emitting diode (OLED), a flexible display, and a 3D display.

According to a type of the apparatus 100, two or more of the displays 130 may be provided. The touch screen may be configured to detect a position, an area, and a pressure of a touch. Also, the touch screen may be configured to detect a proximity touch as well as a real touch.

The control unit 140 may control the image obtaining unit 110, the detecting unit 120, and the display unit 130. Also, the control unit 140 may control the user input unit 150 and the storage unit 160.

The control unit 140 may correct the ultrasound image based on information about the different sound speeds of the plurality of layers. For example, the control unit 140 may correct the ultrasound image by adjusting the delay curve according to the different sound speeds of the plurality of layers.

The user input unit 150 generates input data for controlling an operation of the apparatus 100. The user input unit 150 may include one or more of a keypad, a dome switch, a touchpad (constant voltage/constant capacitance), a jog wheel, and a jog switch. In particular, if the touchpad is combined with the display unit 130 as described above, the combination may be referred to as a touch screen.

The user input unit 150 may receive location information of the plurality of layers having the different sound speeds from the outside. The location information of the plurality of layers having the different sound speeds may include information about boundary lines, boundary surfaces, or boundary regions of the plurality of layers or information about coordinates of the plurality of layers.

The user input unit 150 may receive information about a diagnosed portion from the outside. Examples of the diagnosed portion may include an abdomen, a heart, a brain, an embryo, a breast, a womb, and a thyroid gland.

The storage unit 160 may store a program used by the control unit 140, and temporarily store input/output data. For example, the storage unit 160 may store information about the delay curve according to each sound speed, the corrected ultrasound image, and the ultrasound echo signals.

The storage unit 160 may include at least one type of storage medium selected from the group consisting of a flash memory, a hard disk, a multimedia card micro-type memory, a card-type memory (for example, an SD or XD memory), a random access memory (RAM), a static random access memory (SRAM), a read-only memory (ROM), an electrically erasable programmable read-only memory (EEPROM), a programmable read-only memory (PROM), a magnetic memory, and an optical disk. Also, the apparatus 100 may perform a web storage operation that is a storage function of the storage unit 160 on the Internet.

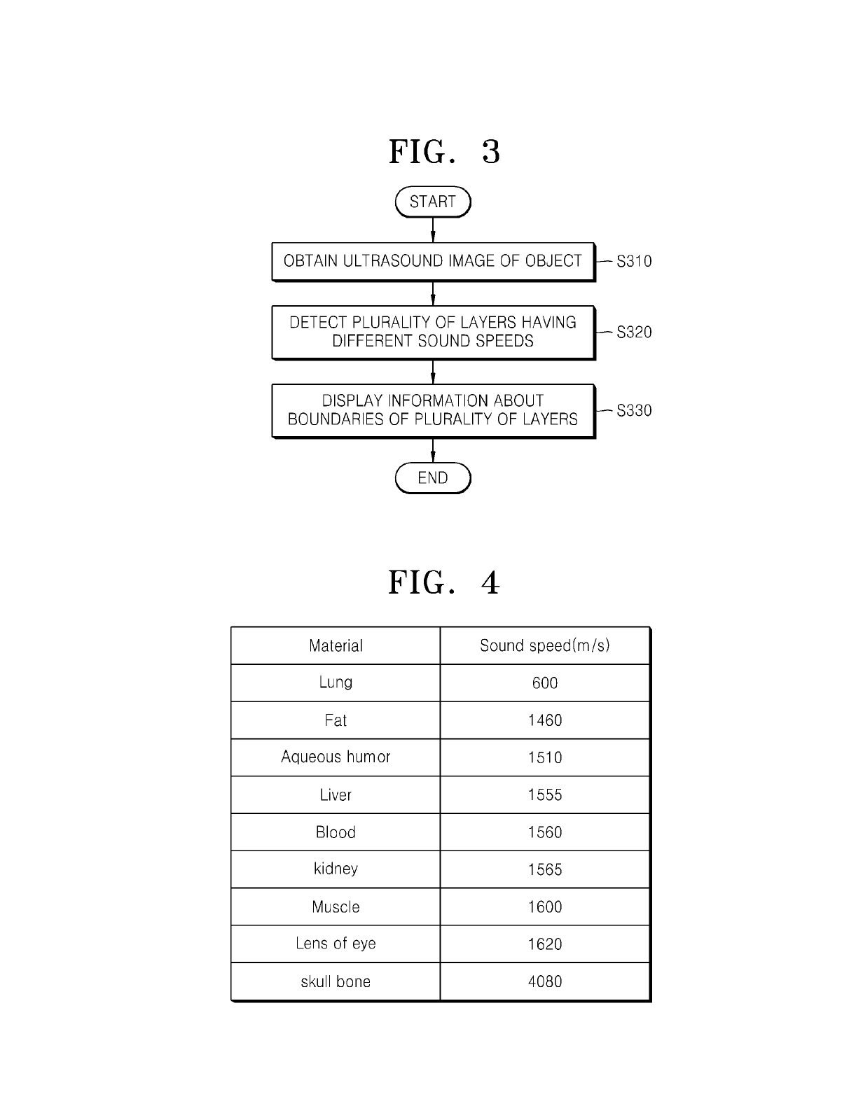

A method of displaying information about boundaries of a plurality of layers having different sound speeds by using the apparatus 100 will be explained with reference to FIG. 3.

FIG. 3 is a flowchart illustrating a method of providing an ultrasound image, according to an embodiment of the present invention.

Referring to FIG. 3, the method includes operations sequentially performed by the apparatus 100 of FIGS. 1 and 2. The description of the apparatus 100 of FIGS. 1 and 2 may apply to the method of FIG. 3 which is also about a method of controlling an output voltage of an ultrasound signal.

Referring to FIG. 3, in operation S310, the apparatus 100 may obtain an ultrasound image of an object. The apparatus 100 may directly generate the ultrasound image.

In this case, the apparatus 100 may generate the ultrasound image by receiving ultrasound echo signals from the object and applying a delay curve to the ultrasound echo signals.

In a body, a sound speed is a very important parameter in forming an ultrasound image. As shown in FIG. 4, sound speeds measured from different human tissues are different from one another. Referring to FIG. 4, a layer having a lowest sound speed is a lung. The reason why the lung has the lowest sound speed is that the lung is an alveolus tissue filled with air. Most human tissues have sound speeds between 1500 m/s to 1600 m/s. Fat tissue has a sound speed lower than that of a soft tissue, and muscle tissue has a sound speed higher than that of the soft tissue. An average sound speed of the soft tissue (including the lung) is about 1540 m/s.

Accordingly, the apparatus 100 may generate an ultrasound image by applying a delay curve at the average sound speed of 1540 m/s.

In operation S320, the apparatus 100 may detect a plurality of layers having different sound speeds based on the ultrasound image obtained in operation S310. The apparatus 100 may detect the plurality of layers having the different sound speeds based on ultrasound echo signals received from the object.

Alternatively, the apparatus 100 may detect the plurality of layers having the different sound speeds based on a brightness difference of the ultrasound image.

In this case, the apparatus 100 may detect the plurality of layers having the different sound speeds by receiving location information of the plurality of layers having the different sound speeds from the outside and further considering the location information.

For example, a user may select a predetermined region including the plurality of layers having the different sound speeds in a displayed ultrasound image according to the brightness difference of the ultrasound image. In this case, the apparatus 100 may detect the plurality of layers having the different sound speeds in a predetermined range based on the predetermined region selected by the user. The apparatus 100 may obtain information about boundaries of the plurality of layers having the different sound speeds.

Accordingly, since the apparatus 100 may detect the plurality of layers having the different sound speeds and the boundaries thereof in the predetermined region selected by the user, the accuracy of the information about the boundaries of the plurality of layers having the different sound speeds may be improved.

The apparatus 100 may detect the plurality of layers having the different sound speeds by receiving information about a diagnosed portion from the outside and considering the information about the diagnosed portion.

For example, if the diagnosed portion is an abdomen, the user may input information indicating that the diagnosed portion is an abdomen to the apparatus 100. In general, since an abdomen consists of a fat layer and a soft tissue layer, the detecting unit 120 may more easily detect the fat layer and the soft tissue layer having different sound speeds.

In operation S330, the apparatus 100 may display the information about the boundaries of the plurality of layers. The apparatus 100 may display the information about the boundaries in such a manner that the information about the boundaries overlaps with the ultrasound image.

The apparatus 100 may display the information about the boundaries of the plurality of layers in various ways by using lines, colors, coordinates, and figures.

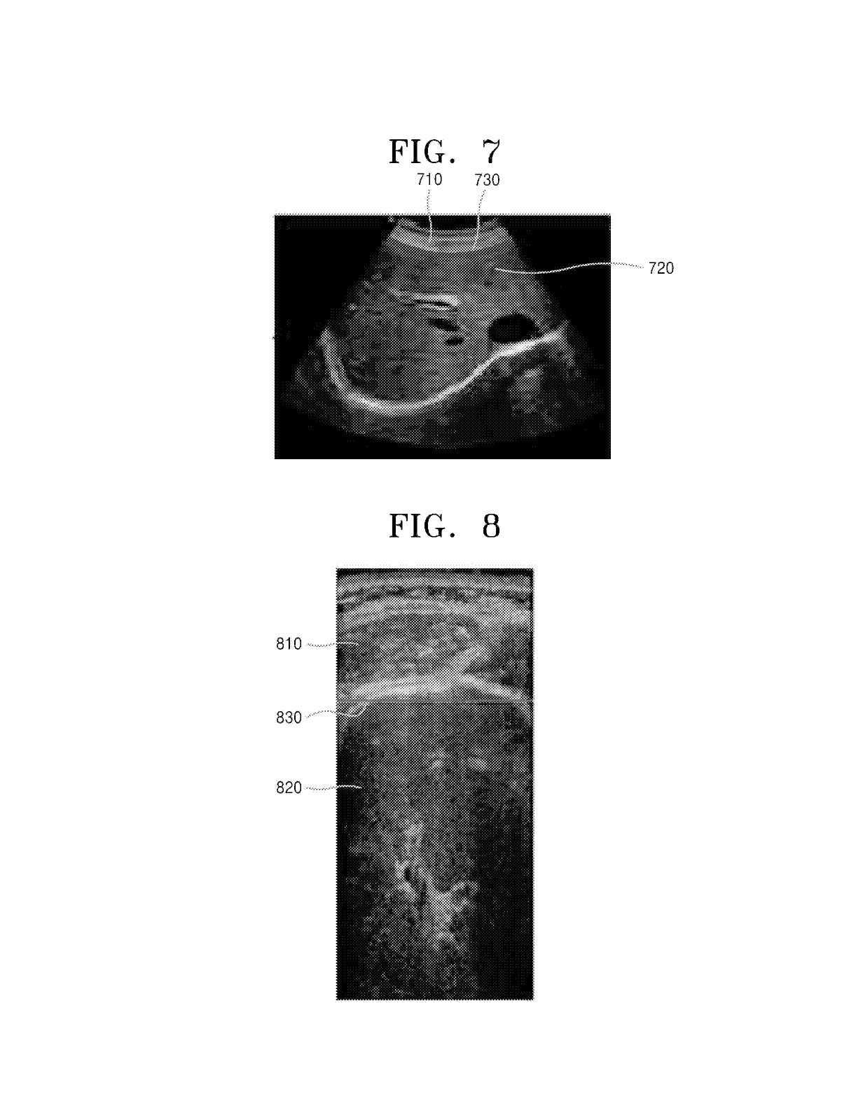

For example, the apparatus 100 may detect a fat layer 710 and a soft tissue layer 720 of FIG. 7 by using the method of FIG. 3. Since the fat layer 710 has a sound speed of 1460 m/s and the soft tissue layer 720 has an average sound speed of 1540 m/s, there is a difference in sound speed between the fat layer 710 and the soft tissue layer 720.

In this case, the apparatus 100 may display a boundary between the fat layer 710 and the soft tissue layer 720 with an auxiliary line 730.

Accordingly, the user may easily distinguish the fat layer 710 from the soft tissue layer 720 having different sound speeds in a displayed ultrasound image.

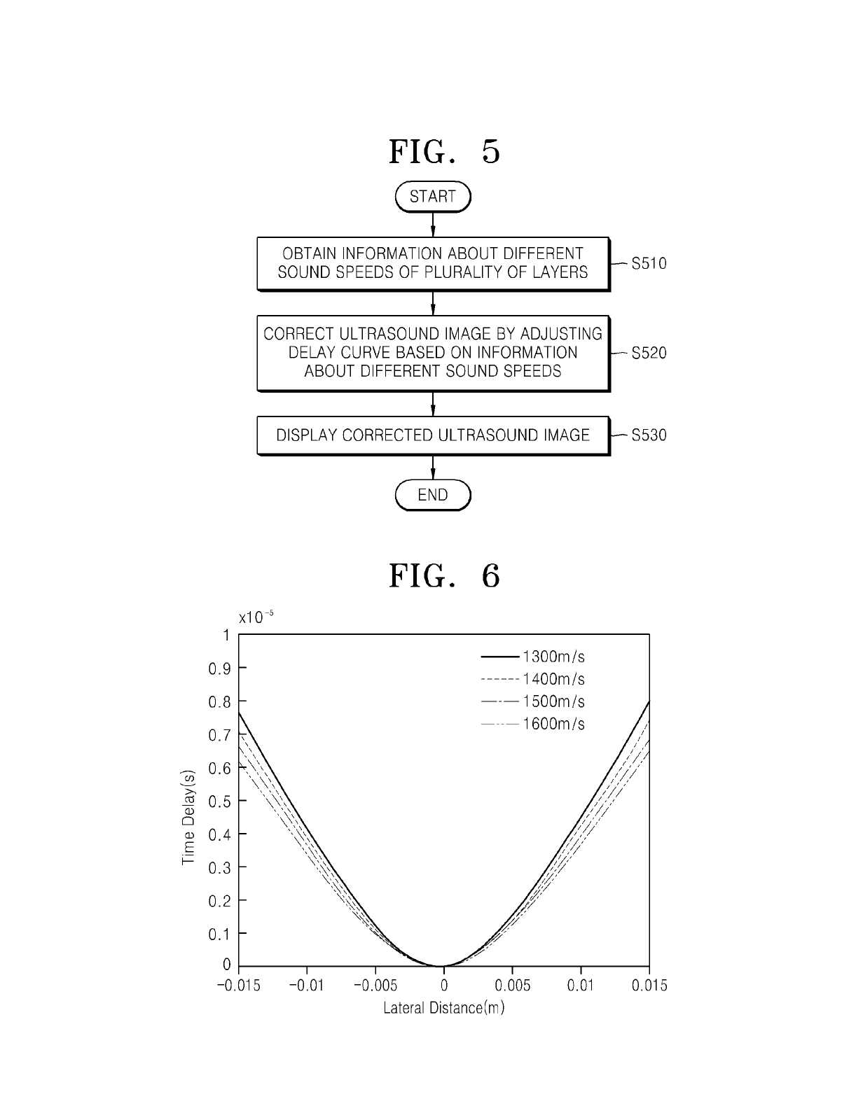

FIG. 5 is a flowchart illustrating a method of correcting an ultrasound image, according to an embodiment of the present invention.

Referring to FIG. 5, in operation S510, the apparatus 100 may obtain information about the different sound speeds of the plurality of layers. The information about the different sound speeds of the plurality of layers may include information about a tissue component, a sound speed corresponding to each layer, and a position (e.g., a depth or coordinates on a scan line) of each layer.

For example, as shown in FIG. 4, if a detected layer is a fat layer, a sound speed may be 1460 m/s and if a detected layer is a muscle layer, a sound speed may be 1600 m/s. As shown in FIG. 6, the apparatus 100 may obtain information indicating that a fat layer is close to a probe and a soft tissue is disposed under the fat layer.

In operation S520, the apparatus 100 may correct the ultrasound image based on the information about the different sound speeds of the plurality of layers. The apparatus 100 may correct the ultrasound image by adjusting the delay curve based on the information about the different sound speeds of the plurality of layers.

In general, a depth of an object, a time delay, and a delay curve are calculated based on an average sound speed (i.e., 1540 m/s) of a soft tissue. However, as shown in FIG. 6, a delay curve varies according to a sound speed. Accordingly, if an ultrasound image is generated by applying 1540 m/s that is an average sound speed, a sensitivity of the ultrasound image is reduced.

For example, although a sound speed of a fat layer is 1460 m/s, since a delay curve corresponding to an average sound speed is used, the quality of an ultrasound image is degraded as a thickness of the fat layer increases.

Accordingly, the apparatus 100 adjusts the delay curve based on the information about the different sound speeds of the plurality of layers.

For example, if one of the plurality of layers is a fat layer and the fat layer is present from a depth of 0 cm to a depth of 5 cm, the apparatus 100 may select a delay curve corresponding to a sound speed (i.e., 1460 m/s) of the fat layer and correct the ultrasound image from the depth of 0 cm to the depth of 5 cm to obtain a corrected ultrasound image.

In operation S530, the apparatus 100 may display the corrected ultrasound image.

Accordingly, a lateral resolution and a contrast of the ultrasound image may be increased. The term “lateral resolution” herein refers to the ability to distinguish two adjacent reflectors that are positioned perpendicular to the axis of an ultrasound beam.

FIG. 8 is an image manually illustrating a boundary between a plurality of layers having different sound speeds, according to an embodiment of the present invention.

A user may input a boundary between a plurality of layers 810 and 820 having different sound speeds through the user input unit 150. In this case, the apparatus 100 displays, on an ultrasound image, information about the boundary between the plurality of layers 810 and 820 having the different sound speeds received from the user. The information about the boundary between the plurality of layers 810 and 820 having the different sound speeds received from the user may be displayed with an auxiliary line 830.

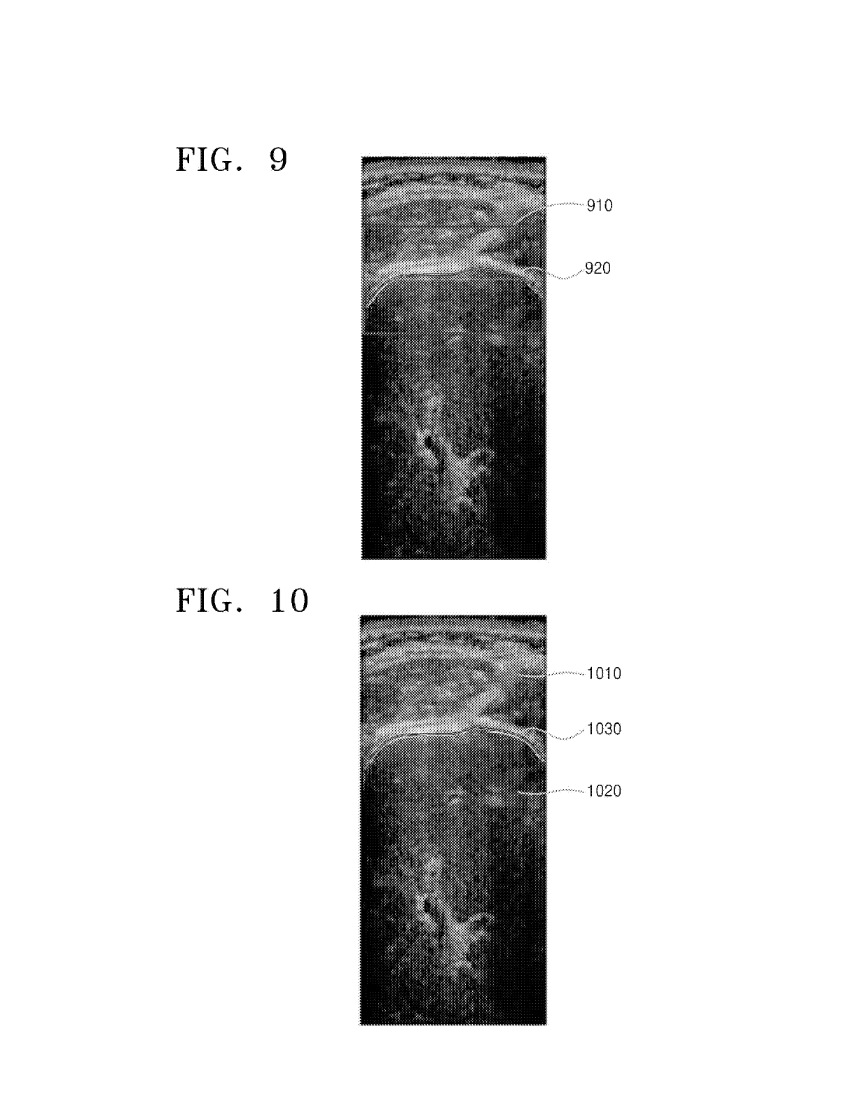

FIG. 9 is an image semi-automatically illustrating a boundary between a plurality of layers having different sound speeds, according to an embodiment of the present invention.

A user may input location information of the plurality of layers having the different sound speeds through the user input unit 150. That is, as shown in FIG. 9, the user may select a specific region 910 including the boundary between the plurality of layers having the different sound speeds through the user input unit 150.

In this case, the apparatus 100 may detect information 920 about the boundary between the plurality of layers having the different sound speeds and the plurality of layers having the different sound speeds based on the specific region 910 selected by the user. The apparatus 100 may display the information 920 about the boundary between the plurality of layers in such a manner that the information 920 overlaps with the ultrasound image.

Since the apparatus 100 detects the boundary between the plurality of layers by using a predetermined algorithm based on the user's input about the boundary between the plurality of layers, the apparatus 100 may more accurately display to the user the information 920 about the boundary between the plurality of layers.

FIG. 10 is an image automatically illustrating a boundary between a plurality of layers having different sound speeds, according to an embodiment of the present invention.

The apparatus 100 may automatically detect a plurality of layers 1010 and 1020 having different sound speeds based on a brightness difference of an ultrasound image or ultrasound echo signals received from an object. Also, the apparatus 100 may generate information 1030 about a boundary between the plurality of layers 1010 and 1020 and automatically display the information 1030 to a user.

According to the present invention, since information about boundaries of a plurality of layers having different sound speeds is displayed to a user, the information may be used as reference data during diagnosis and the quality of an image generated due to differences in sound speed between the plurality of layers may be improved.

The method according to the present invention may be implemented in a program command form that may be executed by various computer elements, and recorded on a computer-readable recording medium. The computer-readable recording medium may include program commands, data files, data structures, or a combination thereof. The program commands recorded on the computer-readable recording medium may be specially designed and constructed for the inventive concept or may be known to and usable by one of ordinary skill in a field of computer software. Examples of the computer-readable recording medium include magnetic media such as a hard disk, a floppy disk, and a magnetic tape, optical media such as a compact disk-read-only memory (CD-ROM) and a digital versatile disk (DVD), magneto-optical media such as a floptical disk, and hardware devices for storing and executing program commands such as a ROM and a flash memory. Examples of the program commands include machine codes made by a compiler and high-level language codes that may be executed by a computer by using an interpreter.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of providing an ultrasound image, the method comprising:
obtaining an ultrasound image of an object;
detecting a plurality of layers having different sound speeds based on the ultrasound image; and
displaying information about boundaries of the plurality of layers.

2. The method of claim 1, wherein the obtaining of the ultrasound image comprises:
receiving ultrasound echo signals from the object; and
generating the ultrasound image by applying a delay curve to the ultrasound echo signals.

3. The method of claim 2, wherein the detecting of the plurality of layers having the different sound speeds comprises detecting the plurality of layers having the different sound speeds based on the ultrasound echo signals received from the object.

4. The method of claim 1, wherein the detecting of the plurality of layers having the different sound speeds comprises detecting the plurality of layers having the different sound speeds based on a brightness difference of the ultrasound image.

5. The method of claim 1, wherein the detecting of the plurality of layers having the different sound speeds comprises:
receiving location information of the plurality of layers having the different sound speeds from the outside; and
detecting the plurality of layers having the different sound speeds by further considering the location information.

6. The method of claim 1, wherein the detecting of the plurality of layers having the different sound speeds comprises:
receiving information about a diagnosed portion from the outside; and
detecting the plurality of layers having the different sound speeds by further considering the information about the diagnosed portion.

7. The method of claim 1, further comprising:
correcting the ultrasound image based on information about the different sound speeds of the plurality of layers to obtain a corrected ultrasound image; and
displaying the corrected ultrasound image.

8. The method of claim 7, wherein the correcting of the ultrasound image comprises adjusting a delay curve based on the information about the different sound speeds of the plurality of layers.

9. An apparatus for providing an ultrasound image, the apparatus comprising:
an image obtaining unit that obtains an ultrasound image of an object;
a detecting unit that detects a plurality of layers having different sound speeds based on the ultrasound image;
a display unit that displays information about boundaries of the plurality of layers; and
a control unit that controls the image obtaining unit, the detecting unit, and the display unit.

10. The apparatus of claim 9, wherein the image obtaining unit comprises:
a receiving unit that receives ultrasound echo signals from the object; and
an image processing unit that generates the ultrasound image by applying a delay curve to the ultrasound echo signals.

11. The apparatus of claim 10, wherein the detecting unit detects the plurality of layers having the different sound speeds based on the ultrasound echo signals received from the object.

12. The apparatus of claim 9, wherein the detecting unit detects the plurality of layers having the different sound speeds based on a brightness difference of the ultrasound image.

13. The apparatus of claim 9, further comprising a user input unit that receives location information of the plurality of layers having the different sound speeds from the outside,
wherein the detecting unit detects the plurality of layers having the different sound speeds by further considering the location information.

14. The apparatus of claim 9, further comprising a user input unit that receives information about a diagnosed portion from the outside,
wherein the detecting unit detects the plurality of layers having the different sound speeds by further considering the information about the diagnosed portion.

15. The apparatus of claim 9, wherein the control unit corrects the ultrasound image based on information about the different sound speeds of the plurality of layers to obtain a corrected ultrasound image,
wherein the display unit displays the corrected ultrasound image.

16. The apparatus of claim 15, wherein the control unit corrects the ultrasound image by adjusting a delay curve based on the information about the different sound speeds of the plurality of layers.

17. A computer-readable recording medium having embodied thereon a program for executing the method of claim 1.
